(19) United States
(12) Patent Application Publication
(10) Pub. No.: US 2010/0247656 A1
(43) Pub. Date: Sep. 30, 2010

(54) Method for in situ Generation of Molecular Iodine on Mucus Membranes Using Nanoparticles

(76) Inventor: James C. Richards, Sudbury, MA (US)

Correspondence Address: CLIFFORD H. KRAFT, 320 ROBIN HILL DR., NAPERVILLE, IL, 60540, US

(21) Appl. No.: 12/731,233

(22) Filed: Mar. 25, 2010

Related U.S. Application Data
(60) Provisional application No. 61/163,205, filed on Mar. 25, 2009.
(60) Provisional application No. 61/163,885, filed on Mar. 27, 2009.
(60) Provisional application No. 61/184,286, filed on Jun. 4, 2009.

(51) Int. Cl. A61K 33/18; A61K 9/14; A61K 33/42

(52) U.S. Cl. 424/489; 424/605; 424/668; 977/775; 977/915

(57) ABSTRACT

Nano-particles created using a biodegradable chemical backbone structure suitably designed to permit creation of at least two populations of nano-particles that contain chemical compositions. A first population of particles can contain iodide (I⁻) and additional compounds including a pH buffer and iodate (IO₃⁻). A second population of particles can contain compounds that will create a microenvironment of pH of 5<-<7 immediately surrounding the second population particle as it is resides on the surface of mucous membranes and cells found in the sinuses and upper airways or on any mucous membrane. The two particle populations are maintained in a dry state for delivery into the nasal passages. When they become hydrated, they now gain an affinity to each other as an activation event, and particles from the two populations residing in proximity to each other diffuse their chemical components into the microenvironment. The pH within the microenvironment surrounding the particles becomes <7.0 with a preferred value of around 6.4. Iodide and iodate react under the mildly acid microenvironment condition to form molecular iodine, I₂ in situ. The particles in association create I₂ nano-particle factories that persistently generate I₂ over time and thereby sustain an anti-microbial/anti-infective environment within the nasal cavity, sinuses and upper airways or on any other mucus membrane.

This application is related to and claims priority from U.S. Provisional Patent applications 61/163,205 filed Mar. 25, 2009 and 61/163,885 filed Mar. 27, 2009. Applications 61/163,205 and 61/163,885 are hereby incorporated by reference.

### BACKGROUND

1. Field of the Invention

The present invention relates generally to the field of killing bacterial, viral and fungal and more particularly to an in situ technique for the generation of molecular iodine in the upper respiratory passages and on other mucus membranes using nano-particle factories.

2. Description of the Prior Art

Prior art methods of killing bacteria, viruses and fungi generally require application of an agent to a particular site. While it has long been known in the art that molecular iodine can kill bacteria, viruses and fungi, it has been impractical, ineffective (and perhaps unsafe) to simply apply any of various iodine solutions directly into the nose or mouth. Because of the tendency of molecular iodine to dissipate when exposed to air, it would be very advantageous if the molecular iodine could be generated in situ rather than applied.

### SUMMARY OF THE INVENTION

The present invention employs nano-particles created using a biodegradable chemical backbone structure suitably designed to permit creation of at least two populations of nano-particles that contain chemical compositions. A first population of particles can contain iodide (I⁻) and additional compounds including a pH buffer and iodate (IO₃⁻). A second population of particles can contain compounds that will create a microenvironment of pH of 5<-<6 immediately surrounding the second population particle as it is resides on the surface of mucous membranes and cells found in the sinuses and upper airways. The two particle populations are maintained in a dry state for delivery into the nasal passages. The particles are evenly dispersed and do not recognize each other in the dry state. However, when they become hydrated, they now gain an affinity to each other as an activation event. Particles from the two populations residing in proximity to each other diffuse their chemical components into the microenvironment. The pH within the microenvironment surrounding the particles becomes <7.0 e.g., around 6.4. Iodide and iodate react under the mildly acid microenvironment condition to form molecular iodine, I₂ in situ. The particles in association create I₂ nano-particle factories that persistently generate I₂ over time and thereby sustain an anti-microbial/anti-infective environment within the nasal cavity, sinuses and upper airways or on any other mucus membrane.

### DESCRIPTION OF THE FIGURES

Attention is directed to a figure that helps understand features of the present invention.

FIG. 1a is a schematic of the two-population particles in the dry state.

FIG. 1b is a schematic of the system in the wet state showing the production of molecular iodine.

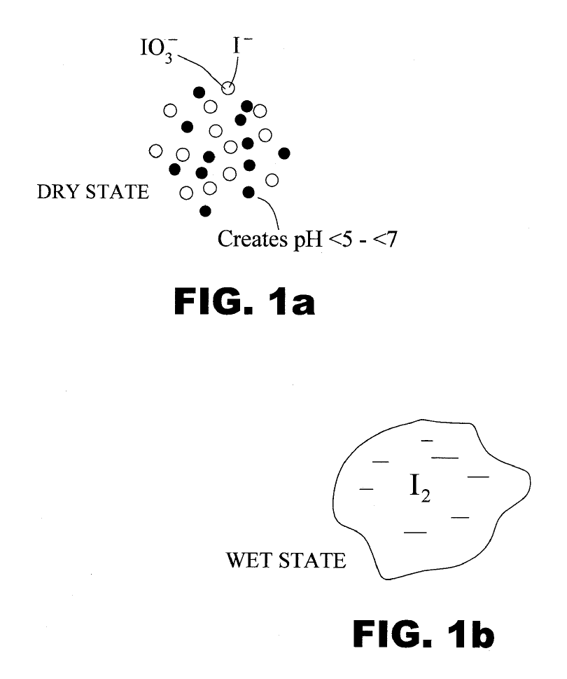

Several illustrations have been provided to aid in understanding the invention. The scope of the present invention is not limited by what is shown in the Figures.

### DESCRIPTION OF THE INVENTION

The present invention employs nano-particles created using cross-linked polysaccharide, poly (lactide-co-glycolide) or other biodegradable chemical backbone structures suitably designed to permit creation of at least two populations of nano-particles that contain chemical compositions. A preferred embodiment has two populations of particles herein described as A and B. While two populations is the preferred method of practicing the invention, any number of populations is within the scope of the present invention.

In a preferred embodiment, population A particles can contain iodide (I⁻) and additional compounds including a pH buffer and iodate (IO₃⁻). Population B particles can contain compounds that will create a microenvironment of pH of 5-6 or <5-<7 immediately surrounding the B particle as it is resides on the surface of mucous membranes and cells found in the sinuses and upper airways. The A and B particle populations are maintained in a dry state for delivery into the nasal passages. The particles are evenly dispersed and do not recognize each other in the dry state. However, when they become hydrated, as when they become attached to cell surfaces in the sinuses or upper airways, they now gain an affinity to each other as an activation event. A and B particles residing in proximity to each other diffuse their chemical components into the microenvironment. The pH within the microenvironment surrounding the particles becomes <7.0 e.g., around 6.4. Iodide and iodate react under the mildly acid microenvironment condition to form molecular iodine, I₂ in situ according to the following equation:

IO₃⁻+5I⁻+6H⁺<->>>3I2+3H₂0

The A and B particles in association create I₂ nano-particle factories that persistently generate I₂ over time and thereby sustain an anti-microbial/anti-infective environment within the nasal cavity, sinuses and upper airways or on any other mucus membrane. The I₂ generated in situ immediately reacts with target sites (proteins and membranes) on bacteria, virus, and fungi as well as general mucous, epithelial cells and other reactive sites. Iodinated microbes become inactivated. A further advantage of the present invention is that localized I₂ generation creates pockets of more concentrated I₂ that will break down and dissociate mucous accumulation in the sinuses and nasal passages and therefore promote sinus drainage and unmask pathogens growing in the mucous layer making them available to the killing action of the I₂.

A possible source of iodide and iodate for the first population particles is potassium iodide and potassium iodate. These salts have the property of being highly water-soluble, yet stable in the dry state. Sodium and other water-soluble iodides and iodates will also work. A possible choice for a compound that will produce a pH between <5-<7 is any water-soluble, biologically safe, slightly acid compound that has a stable dry state such as a phosphate or other slightly acid inorganic or organic salt. In some cases, the backbone alone will provide a proper pH.

Several descriptions and illustrations have been presented to aid in understanding the features of the present invention. One with skill in the art will realize that numerous changes and variations are possible without deviating from the spirit of the invention. Each of these changes and variations is within the scope of the present invention.

I claim:

1. A method of killing bacteria, viruses and fungi on a mucus membrane by in-situ generation of molecular iodine comprising:
providing a first population of biodegradable nano-particles that contain iodide and iodate in a dry state;
providing a second population of biodegradable nano-particles in a dry state that contain a compound that when hydrated will create a micro-environment of <pH 5 to <pH 7;
mixing said first and second populations of nano-particles in a dry state, wherein said first and second populations do not chemically react in said dry state, and wherein when said first and second populations of nano-particles are introduced onto a mucus membrane, said nano-particles become hydrated and chemically react to form molecular iodine on said mucus membrane.

2. The method of claim 1 wherein said mucus membrane is a nasal passage.

3. The method of claim 1 wherein said first population of nano-particles further contains a pH buffer compound.

4. The method of claim 1 wherein said second population of nano-particles contains a phosphate.

5. The method of claim 1 wherein said first population of nano-particles contains potassium iodate and potassium iodide.

6. A method of killing bacteria, viruses and fungi in a nasal passage by in-situ generation of molecular iodine comprising:
providing a first population of biodegradable nano-particles in a dry state that contain iodide and iodate;
providing a second population of biodegradable nano-particles in a dry state that contain a compound that will create a micro-environment of around pH 5-7 when hydrated;
mixing said first and second populations of nano-particles in a dry state wherein said first and second populations do not chemically react in said dry state, and wherein when said first and second populations of nano-particles are introduced into a nasal passage or other mucus membrane, said nano-particles become hydrated and chemically react to form molecular iodine in said nasal passage or on said mucus membrane.

7. The method of claim 6 wherein said first population of nano-particles further contains a pH buffer compound.

8. The method of claim 6 wherein said second population of nano-particles contains a phosphate.

9. The method of claim 6 wherein said first population of nano-particles contains potassium iodate and potassium iodide.

10. A method of killing bacteria, viruses and fungi on a mucus membrane by in-situ generation of molecular iodine comprising:
providing a first population of biodegradable nano-particles that contain potassium or sodium iodide and potassium or sodium iodate in a dry state; providing a second population of biodegradable nano-particles that contain a compound in a dry state which when hydrated will create an environment of pH from around 5-7; wherein, when said first and second particles are introduced onto a mucus membrane they produce molecular iodine.

11. The method of claim 10 wherein said introduced second population of particles create an environment of pH of around 6.4.

12. The method of claim 10 wherein said first population of nano-particles further contains a pH buffer compound.
